(19) United States
(12) Patent Application Publication
(10) Pub. No.: US 2023/0405295 A1
(43) Pub. Date: Dec. 21, 2023

(54) Cathlock System for Proximally Trimmable Catheter

(71) Applicant: Bard Peripheral Vascular, Inc., Franklin Lakes, NJ (US)

(72) Inventors: Brenda L. F. Fedor, Holladay, UT (US); Jason R. Stats, Layton, UT (US); Bradley Forsyth, Sandy, UT (US); Nathan Gooch, Centerville, UT (US); Jessica Mathews Roundy, Riverton, UT (US); Tomas Jordan Cartmill, Midvale, UT (US); Bryon Ray Densley, Rochester, MN (US)

(21) Appl. No.: 18/036,367

(22) PCT Filed: Nov. 20, 2020
(86) PCT No.: PCT/US20/61600

§ 371 Date: May 10, 2023

(51) Int. Cl. A61M 39/12; A61M 39/10

(52) U.S. Cl. CPC A61M 39/12; A61M 2039/1077; A61M 39/1011

(57) ABSTRACT

Embodiments disclosed herein are directed to connections systems including a port, a catheter, a cathlock and an adapter. The adapter can be coupled with a proximal end of the catheter and a cathlock can co-operate with the adapter to secure the catheter to the port in a fluid tight engagement. One of the cathlock or the adapter can threadably engage the port and employ mechanical advantage to radially compress the catheter and ensure a fluid-tight seal. The system can further include an insertion tool configured to facilitate securing the adapter to the catheter or securing the adapter or cathlock to the port. The adapter can be provided as a separate structure or coupled to the cathlock. Engagement of the adapter with the catheter can be signaled with an audible click. Optionally the adapter can include a skirt configured to compress the catheter onto the adapter stem.

### SUMMARY

Briefly summarized, embodiments disclosed herein are directed to a cathlock system for coupling proximally trimmable catheters and ports. Proximally trimmable catheters allow for post-placement sizing of the catheter. When placing a catheter and port assembly, the position of the distal tip of the catheter can be important for the efficacy of the treatment. For example, when placing a catheter within the superior vena cava, if the distal tip of the catheter falls short of the target area, the efficacy of the medicament is reduced. If the distal tip is advanced too far, the distal tip can cause arrhythmia. The distance between the distal tip of the catheter and the port can vary since the distances between the target location, insertion site to the vasculature, and the location of the port can vary between patients and procedures. Estimating the catheter length before placement can lead to errors that result in misplacement of the distal tip.

Proximally trimmable catheters allow for placement of the catheter distal tip at the target location before trimming a proximal portion of the catheter to the correct length. The clinician can then attach the catheter to a subcutaneous port, or similar access device. However, securing the catheter to the port can be challenging. The connection must be leak-proof, especially under high-pressure infusion. Further, manipulating the catheter and port within the confined, wetted environment of a subcutaneous access site can lead to slippage, undue trauma to the access site, or misplacement of the catheter distal tip.

Embodiments disclosed herein are directed to cathlock systems that use mechanical advantage to secure the catheter to the port stem to provide a leak-proof connection even under high pressure.

Disclosed herein is a connection system for a subcutaneous port including a port stem, the connection system including, an adapter having a body and configured for connection to the port stem, and an adapter stem extending from the body and configured for insertion into a lumen of a catheter to connect the adapter to the catheter, and a cathlock including a lumen and configured for sliding over an outer surface of the catheter, the cathlock having threading for engagement with the port stem, wherein engaging the cathlock to the port stem compresses the catheter radially inward onto the adapter.

In some embodiments, the adapter stem is configured to radially expand the catheter lumen to secure the adapter to the catheter in an interference fit. The adapter is formed of a resilient material. The adapter body engages the port stem in one of an interference fit, press fit, snap-fit, or luer-slip fit engagement. The adapter includes a skirt extending longitudinally from the body and disposed annularly about the adapter stem, the skirt configured to elastically deform radially inward to engage the outer surface of the catheter. The skirt is formed of a plurality of fingers extending longitudinally from the body of the adapter and disposed annularly about the adapter stem, the plurality of fingers configured to elastically deform radially inward to engage the outer surface of the catheter. A tip of a finger of the plurality of fingers includes a protrusion extending radially inward and configured to engage a portion of the catheter.

In some embodiments, an outer surface of the skirt includes a threaded portion configured to threadably engage the cathlock. The cathlock includes a body rotatably coupled to a collar, the collar including a lug configured to engage a helical channel disposed on the port, and wherein rotating the collar urges the lug through the helical channel and urges the cathlock in a longitudinal direction. An inner surface of the collar includes a collar locking tooth configured to engage a port locking tooth disposed on the port and to provide an audible or tactile indicator, or to prevent retrograde rotation of the collar. The cathlock body and collar define the cathlock lumen, a lumen diameter at the body includes a first diameter and a lumen diameter at the collar defines a second diameter, larger than the first diameter, the lumen including a tapered portion extending from the second diameter to the first diameter. A portion of the catheter disposed on the adapter stem defines an outer diameter that is greater than the first diameter of the cathlock lumen and less than the second diameter of the cathlock lumen. The cathlock lumen includes a ring extending radially inward from an inner wall thereof, the ring configured to abut against a shoulder of the adapter.

In some embodiments, the port includes a gasket disposed between the port and the adapter and encircling the port stem. In some embodiments, the connection system further includes an insertion tool having a handle, an adapter tool configured to engage a lumen of the adapter, or a spanner head configured to engage the collar of the cathlock. The spanner head of the insertion tool includes a jaw configured to engage a facet of the collar and to facilitate rotation of the collar about the longitudinal axis. In some embodiments, the jaw includes a lip configured to engage an undercut of a collar ridge of the collar and to facilitate rotation of the collar about the longitudinal axis. In some embodiments, a proximal end of the catheter is trimmable. In some embodiments, the adapter includes a pawl configured to engage a recess disposed in the cathlock in a snap-fit engagement to provide an audible signal that the lumen of the catheter is fully engaged with the adapter. The port includes a port locking tooth, and the cathlock includes a cathlock locking tooth, extending from a surface of the cathlock lumen, the port locking tooth configured to engage the cathlock locking tooth in a snap-fit engagement to mitigate retrograde rotation of the cathlock.

Also disclosed is a cathlock system for coupling a catheter to a port including, a port including a port stem and a stem housing encircling a portion of the port stem and defining an opening, the stem including a flared portion, and a cathlock including a body and a collar extending longitudinally therefrom, the collar configured to extend between an outer surface of the port stem and an inner surface of the stem housing opening.

In some embodiments, the port stem includes a first portion defining a first outer diameter, a flared portion defining a second outer diameter greater than the first outer dimeter, and a tapered portion transitioning between the first outer diameter and the second outer diameter. The first outer diameter is the same or slightly smaller than an inner diameter of a lumen of the catheter in an unstressed state and the second diameter is greater than the inner diameter of the lumen of the catheter in an unstressed state. The flared portion is disposed within the stem housing. The cathlock engages the stem housing with one of a threaded engagement, press-fit engagement, interference fit engagement, or luer-slip fit engagement. The cathlock is configured to compress a portion of the catheter between an inner surface of the cathlock lumen and an outer surface of the port stem.

In some embodiments, the catheter includes a coating having a low friction co-efficient. A lumen of the cathlock body defines a first lumen diameter, and a lumen of the cathlock collar defines a second diameter, greater than the first diameter, the first diameter configured to engage the first portion of the port stem, the second diameter configured to engage the flared portion of the port stem. An outer surface of the cathlock includes a facet or a gripping feature configured to facilitate rotation of the cathlock about the longitudinal axis.

Also disclosed is an adapter configured to couple a catheter to a port including, a body defining a circular cross-sectional shape and including a lug disposed on an outer surface thereof configured to engage a helical channel disposed on the port, and an adapter stem extending longitudinally from the body and configured to engage a lumen of a catheter in an interference fit.

In some embodiments, the adapter further includes a cathlock slidably engaged with an outer surface of the catheter and configured to compress a portion of the catheter against the adapter stem. In some embodiments, the adapter further includes a skirt extending longitudinally from the adapter body and encircling the adapter stem, the skirt configured to engage an outer surface of the catheter and elastically deflect to compress a portion of the catheter against the adapter stem. The skirt includes a plurality of fingers configured to elastically deflect to compress a portion of the catheter against the adapter stem. The body is configured to rotate to engage the lug with the helical channel and urge the body in a longitudinal direction. The body is configured to fit within a socket disposed within the port, the helical channel disposed on an inner surface of the socket.

Also disclosed is a method of coupling a catheter to a port including, placing a distal portion of the catheter within a vasculature, trimming a proximal portion of the catheter, urging a stem of a cathlock adapter into a lumen of the catheter, rotating a cathlock to threadably engage a portion of the port, radially compressing the proximal portion of the catheter on to the stem of the cathlock adapter, and longitudinally compressing the cathlock adapter onto the stem of the port.

In some embodiments, urging the stem of the cathlock adapter into the lumen further includes expanding an outer diameter of the proximal portion of the catheter. In some embodiments, the expanded outer diameter of the proximal portion of the catheter is greater than an inner diameter of a distal opening of the cathlock. In some embodiments, urging the stem of a cathlock adapter into a lumen of the catheter further includes one of an interference fit, press fit, or snap-fit engagement. In some embodiments, the method further includes compressing a skirt radially inward to engage an outer surface of the catheter, the skirt extending longitudinally from a body of the cathlock adapter and disposed annularly about the cathlock adapter stem. Rotating the cathlock further includes threadably engaging an outer surface of the skirt. The skirt includes a plurality of fingers extending longitudinally from the body of the cathlock adapter.

In some embodiments, urging the stem of the cathlock adapter into the lumen of the catheter includes engaging an insertion tool having a handle and an adapter tool, with a lumen of the cathlock adapter. Rotating the cathlock includes engaging a spanner head of the insertion tool with a facet of a collar of the cathlock. The adapter is coupled to the cathlock and wherein the adapter engages the cathlock in a snap-fit engagement to provide an audible sound when urging a stem of a cathlock adapter to be fully engaged with a lumen of the catheter.

Also disclosed is a method of coupling a catheter to a port including, stretching a proximal end of a catheter over a flared port stem to engage therewith in an interference fit, engaging an outer surface of a collar of a cathlock with an inner surface of a stem housing, and compressing a wall of the proximal end of the catheter between the cathlock and the flared port stem.

In some embodiments, the flared port stem includes a first portion defining a first outer diameter, and a flared portion defining a second outer diameter, greater than the first outer dimeter, and a tapered portion transitioning between the first outer diameter and the second outer diameter. In some embodiments, engaging the collar with the stem housing includes one of a threaded engagement, press-fit engagement, interference fit engagement, or luer-slip fit engagement.

Also disclosed is a method of coupling a catheter to a port including, engaging an adapter stem with a lumen of the catheter, inserting an adapter body into a port socket, and rotating the adapter body to engage a lug with a helical channel.

In some embodiments, the method further includes slidably engaging a cathlock over a portion of the catheter to compress the catheter onto the adapter stem.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows an exploded view of a port, catheter and cathlock assembly, in accordance with embodiments disclosed herein.

FIG. 1B shows close up detail of a cathlock adapter engaged with a catheter, in accordance with embodiments disclosed herein.

FIGS. 1C-1D show perspective views of a port, catheter and cathlock assembly, in accordance with embodiments disclosed herein.

FIG. 2A shows an exploded view of an insertion tool and a cathlock adapter, in accordance with embodiments disclosed herein.

FIG. 2B shows a perspective view of an insertion tool engaged with a cathlock adapter, in accordance with embodiments disclosed herein.

FIG. 3A shows a perspective view of an insertion tool, in accordance with embodiments disclosed herein.

FIG. 3B shows close up detail of an insertion tool, in accordance with embodiments disclosed herein.

FIGS. 3C-3E show an insertion tool engaged with a cathlock collar, in accordance with embodiments disclosed herein.

FIGS. 3F-3G show a cross-section view of an insertion tool engaged with a cathlock collar, in accordance with embodiments disclosed herein.

FIG. 4A shows a vertical cross-section view of a port, catheter and cathlock assembly, in accordance with embodiments disclosed herein.

FIG. 4B shows a horizontal cross-section view of a port, catheter and cathlock assembly, in accordance with embodiments disclosed herein.

FIG. 4C shows a plan view of a horizontal cross-section of a port, catheter, and cathlock assembly, in accordance with embodiments disclosed herein.

FIG. 4D shows a plan view of a horizontal cross-section of a port, catheter, and cathlock assembly, in accordance with embodiments disclosed herein.

FIG. 5A shows a cross section view of an insertion tool engaged with a cathlock adapter that is coupled with a cathlock, in accordance with embodiments disclosed herein.

FIGS. 5B-5C show close up detail of the adapter and cathlock of FIG. 5A, in accordance with embodiments disclosed herein.

FIG. 5D shows a perspective view of an insertion tool engaged with a cathlock adapter that is coupled with a cathlock, the cathlock being shown in wire frame, in accordance with embodiments disclosed herein.

FIG. 5E shows a cross-section view of an insertion tool engaged with a cathlock adapter that is coupled with a cathlock, in accordance with embodiments disclosed herein.

FIG. 6A shows an exploded, cross section view of a cathlock adapter, cathlock and catheter, in accordance with embodiments disclosed herein.

FIG. 6B shows a side view of a cathlock adapter, in accordance with embodiments disclosed herein.

FIG. 6C shows a cross-section view of a cathlock adapter, in accordance with embodiments disclosed herein.

FIG. 6D shows a side view of a cathlock adapter, in accordance with embodiments disclosed herein.

FIG. 6E shows a cross-section view of a cathlock adapter, in accordance with embodiments disclosed herein.

FIG. 7A shows an exploded view of a port, catheter and cathlock assembly, in accordance with embodiments disclosed herein.

FIG. 7B shows a perspective view of a cathlock, in accordance with embodiments disclosed herein.

FIG. 7C shows a cross-section view of a cathlock, in accordance with embodiments disclosed herein.

FIGS. 8A-8C show cross-section views of an exemplary method of attaching a port, catheter, and cathlock assembly, in accordance with embodiments disclosed herein.

FIGS. 8D-8F show perspective views of an exemplary method of attaching a port, catheter, and cathlock assembly, in accordance with embodiments disclosed herein.

FIG. 9A shows a cross-section exploded view of an adapter, cathlock, and catheter assembly, in accordance with embodiments disclosed herein.

FIGS. 9B-9C show a cross-section exploded view of an adapter, cathlock, and catheter assembly, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

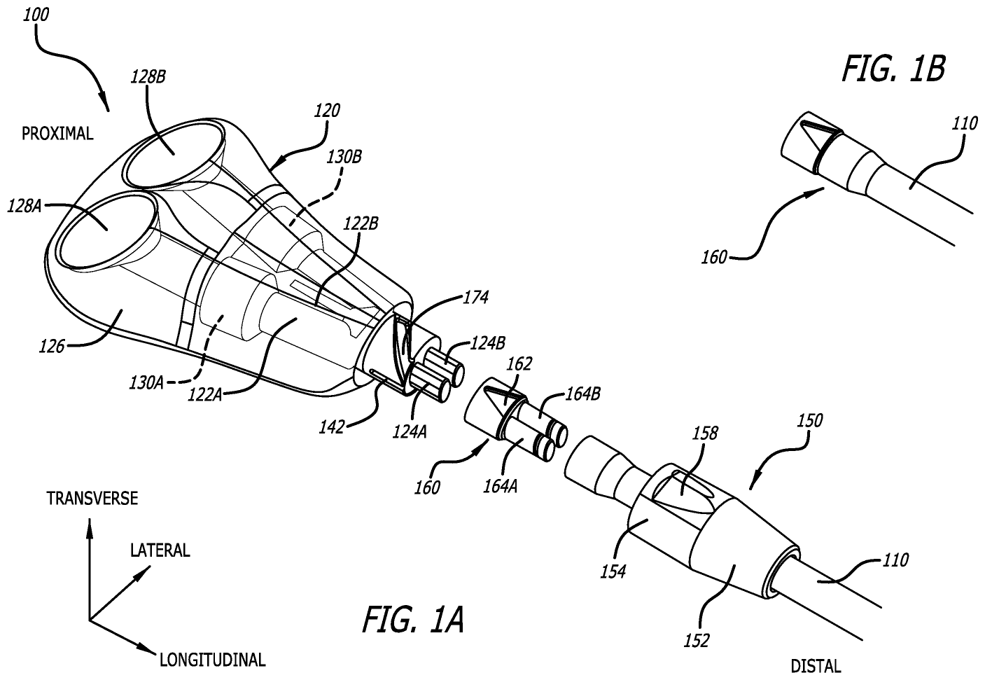

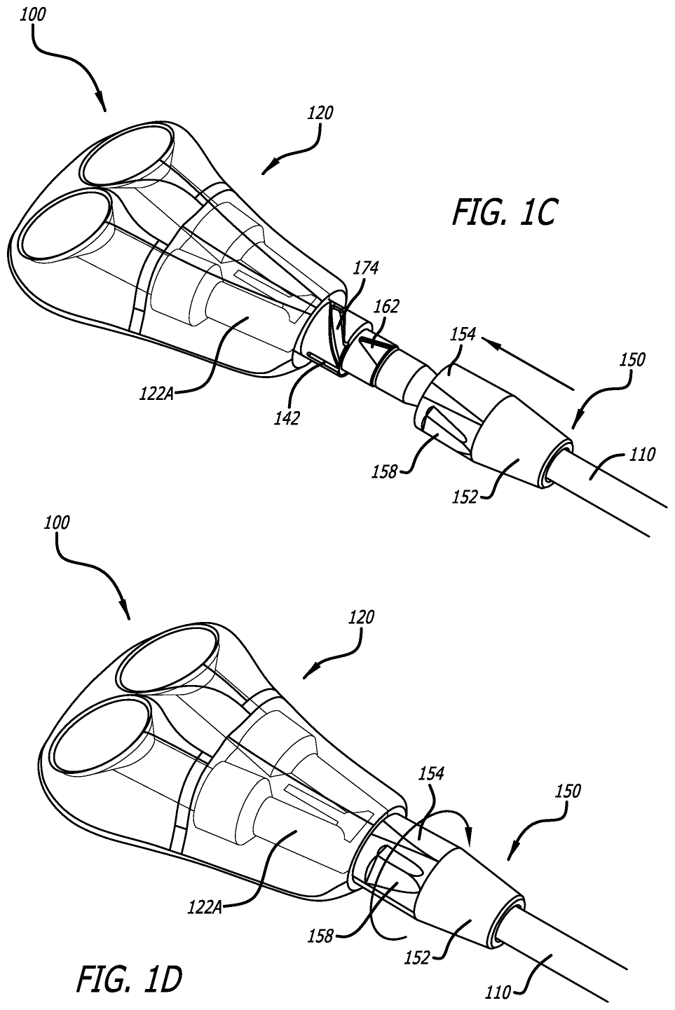

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes. Unless defined otherwise, all technical and scientific terms used herein have the same meaning as commonly understood by those of ordinary skill in the art.

FIGS. 1A-1D shows various views of a connection system (“system”) 100 including a proximally trimmable catheter 110, a port 120, a cathlock 150, and a cathlock adapter 160, in accordance with embodiments disclosed herein. As used herein, the port 120 can be any single lumen or multi-lumen, subcutaneous or supercutanous, medical access device configured to provide fluid access to the catheter 110. The system 100 includes a port 120 fluidly coupled to a proximally trimmable catheter 110 by way of cathlock adapter 160 and secured in place with a cathlock 150. FIG. 1A shows an exploded view of the system 100. FIG. 1B shows close up detail of the cathlock adapter 160 coupled to the proximal end of the catheter 110. FIG. 1C shows the cathlock adapter 160 and catheter 110 assembly coupled with the port stem 124. FIG. 1D shows the cathlock 150 secured to the port 120 to secure the catheter 110 and cathlock adapter 160 to the port 120.

In an embodiment the catheter 110 can define a lumen 112. However, multi-lumen catheters are also contemplated to fall within the scope of the present invention. For example, as shown, the catheter 110 can define a first lumen 112A and a second lumen 112B. In an embodiment, the catheter 110 can be formed of a compliant, trimmable material, such as a plastic, polymer, rubber, silicone, or the like.

In an embodiment, the port 120 can be a medical access device configured to provide fluid communication with the catheter 110. A lumen of the port 120 can be fluidly coupled with a lumen 112 of the catheter 110. As shown, the system 100 includes a dual lumen port 120 fluidly coupled with a dual lumen catheter 110, however it will be appreciated that single lumen assemblies and multi-lumen assemblies are also contemplated to fall within the scope of the present invention.

The port 120 includes a body 122 formed by a similarly shaped first conduit 122A and second conduit 122B that are each in fluid communication with a lumen 112 of the catheter 110. For example, the first conduit 122A can be in fluid communication with a first lumen 112A and the second conduit 122B can be in fluid communication with second lumen 112B. The port body 122 includes a port stem 124 extending distally from a distal end thereof. For example, a first port stem 124A can be in fluid communication a first conduit 122A and a second port stem 124 can be in fluid communication with a second conduit 122B. In an embodiment, the port 120 further includes an outer shell 126 that is overmolded onto a portion of the port body 122. A portion of the outer shell 126 can be formed of a compliant material, such as silicone, or similar suitable material. The first and second conduits 122A, 122B, can include a metal, such as titanium. It will be appreciated that the port body 122, or portions thereof, can include a variety of materials, including metals, thermoplastics, ceramics, etc. Further the first and second conduits 122A, 122B, or portions thereof can be assembled using various joining methods including snap-fitted, press-fit, adhesive, ultrasonic or other welding, interference fit, combinations thereof, or the like.

One of the first conduit 122A or the second conduit 122B can include a substantially funnel-shaped receiving cup 128 for receiving and directing a catheter-bearing needle, or similar such medical device, to operably connect with the port 120. For example a first receiving cup 128A can be coupled to a first conduit 122A and a second receiving cup 128B can be coupled to a second conduit 122B. One of the first conduit 122A or the second conduit 122B can include a valve 130, e.g. a first valve 130A and a second valve 130B, configured to allow a needle to fluidly engage the port and pass distally to provide fluid communication between the needle and the conduit of the port body 122, as well as configured to prevent a proximal fluid flow from the conduit 122.

Further details of embodiments of the port and catheter can be found in U.S. 2019/0232035, filed Apr. 11, 2019, and is herein incorporated by reference in its entirety.

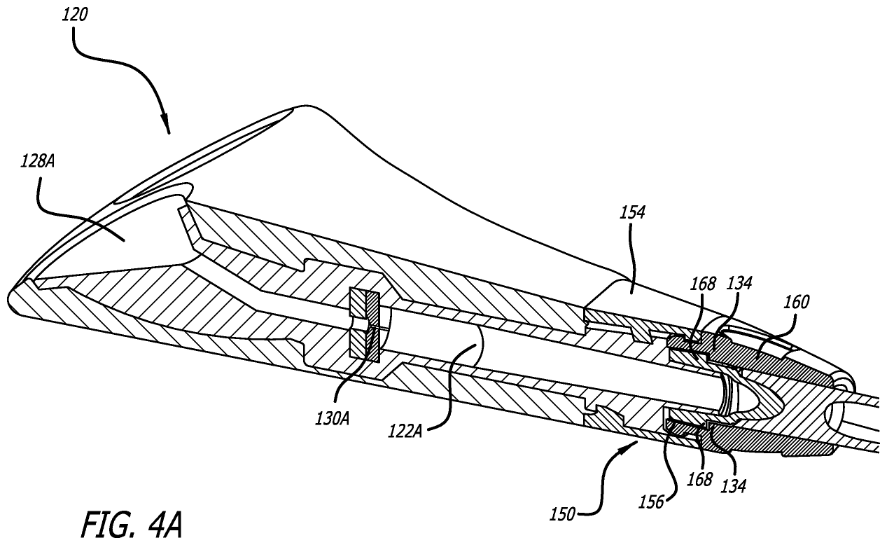

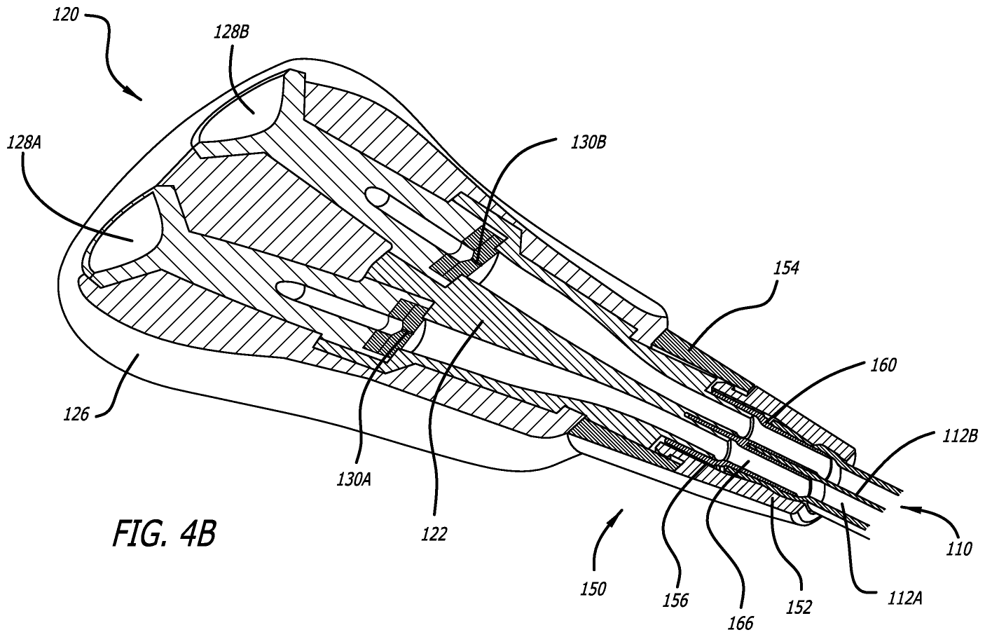

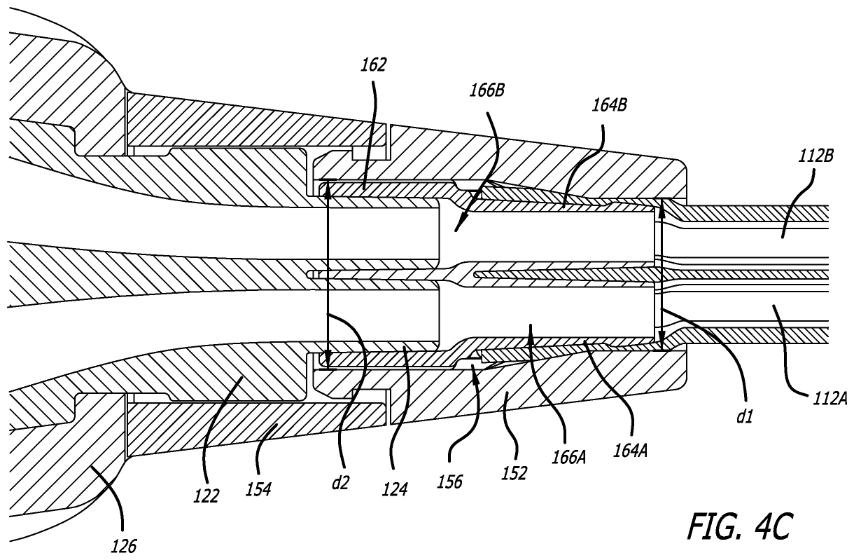

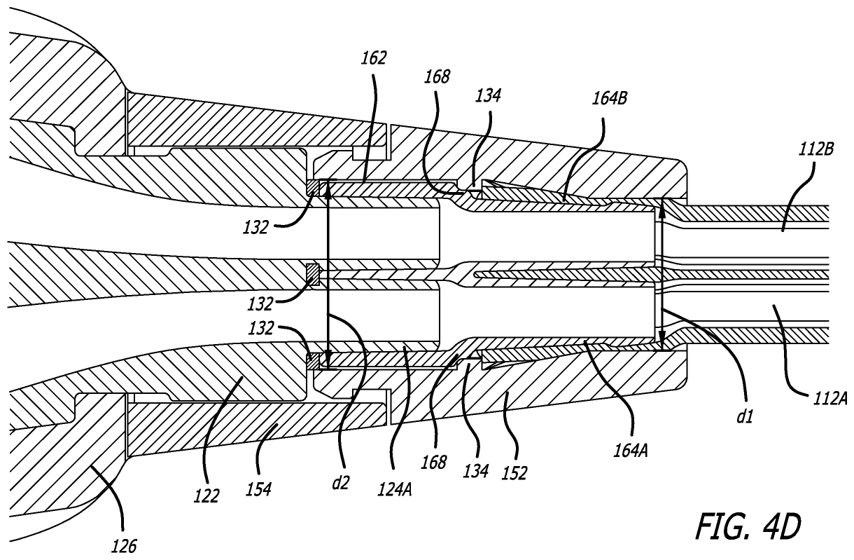

In an embodiment, the cathlock 150 can include a cathlock body 152 and a collar 154 rotatably coupled thereto. The cathlock body 152 can define a substantially cylindrical shape and define a cathlock lumen 156. As shown in FIGS. 4A-4C, the cathlock lumen 156 can define a substantially tapered interior profile where a distal end of the cathlock lumen 156 defines a first inner diameter (d1) and a proximal end defines a second inner diameter (d2), the second diameter (d2) being larger than the first diameter (d1). In an embodiment, the cathlock lumen 156, or portion thereof, can provide a tapered, continuous increase in diameter between the first diameter (d1) and the second diameter (d2), a stepped increase in diameter between the first diameter (d1) and the second diameter (d2), or combinations thereof. In an embodiment, the cathlock 150 can be formed of a substantially rigid or resilient material.

The cathlock collar 154 can be rotatably coupled to the cathlock body 152 and can rotate about the longitudinal axis. In an embodiment, the collar 154 can rotate freely about the longitudinal axis while remaining coupled to the cathlock body 152. In an embodiment, the cathlock collar 154 can rotate about the longitudinal axis through a predetermined arc, for example an arc of between 30° and 720°. However, greater or lesser arcs are also contemplated. In an embodiment, the cathlock collar 154 can include one or more facets 158, or similar gripping feature, configured to engage an insertion tool 180, to facilitate rotating the collar 154 about the longitudinal axis, as described in more detail herein. In an embodiment, the collar 154 can include notches, ridges, ribs, materials with a high friction co-efficient, combinations thereof or the like, also configured to facilitate rotating the collar 154 about the longitudinal axis.

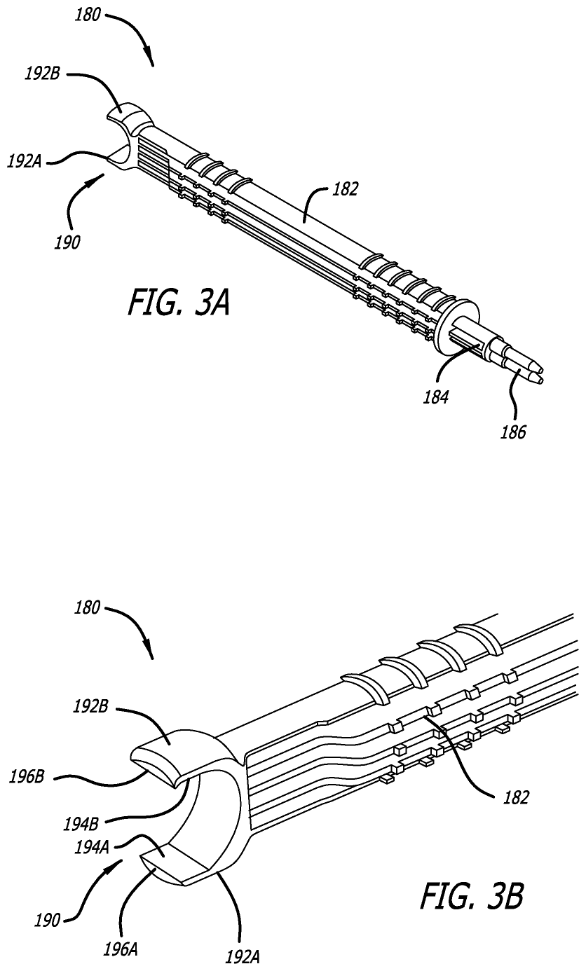

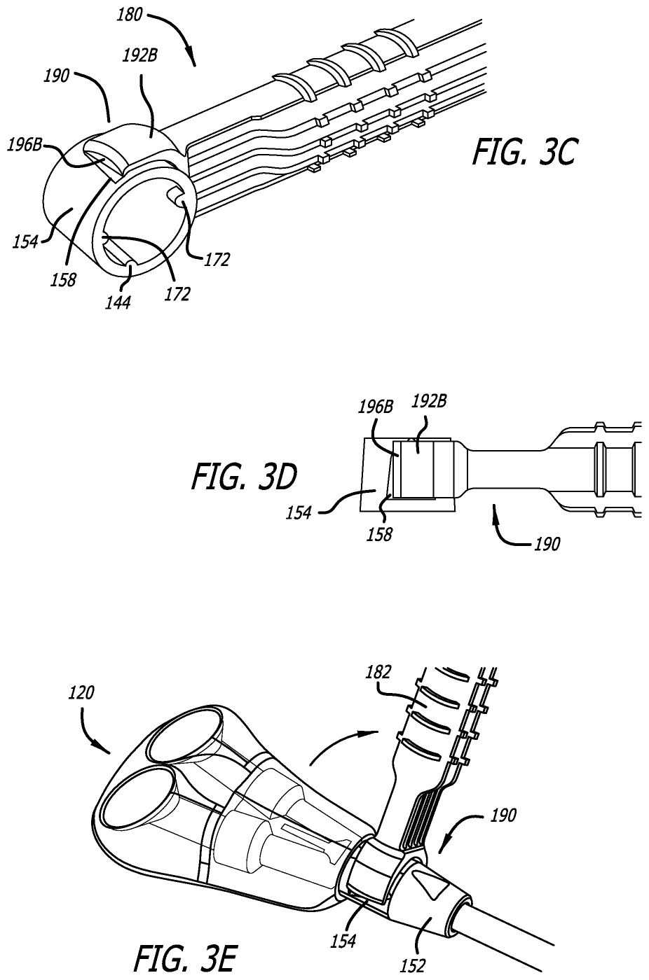

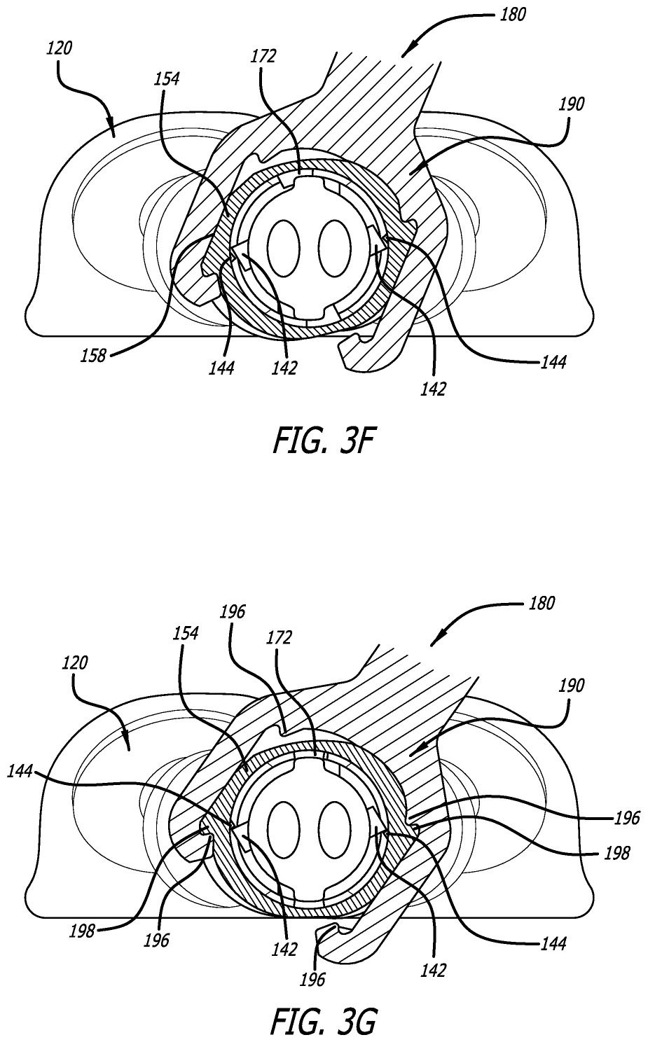

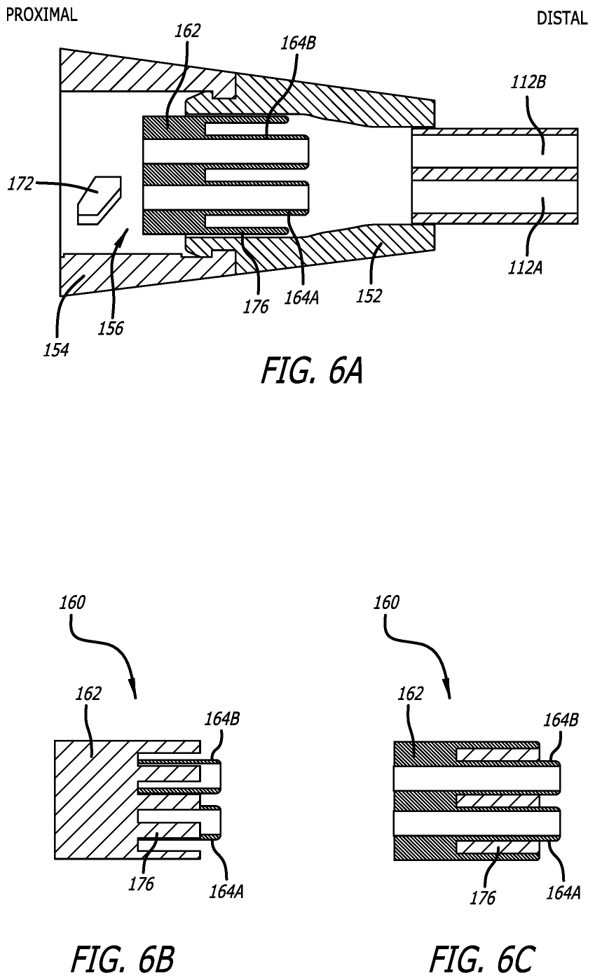

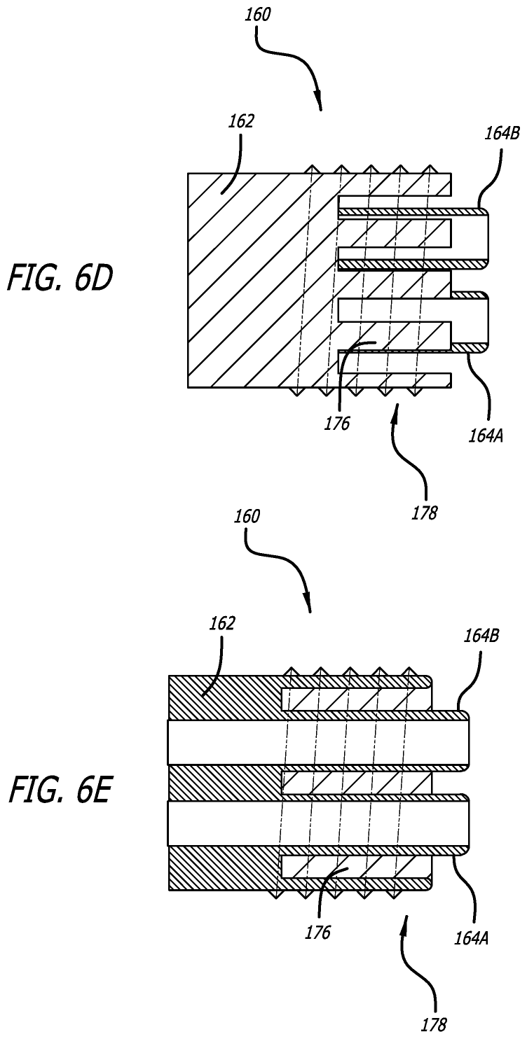

In an embodiment, the cathlock collar 154 can threadably engage the port 120. For example, an inner surface of the cathlock collar 154 can include one or more lugs 172 (see FIGS. 3C, 6A) extending radially inward therefrom, and configured to engage a helical channel 174 disposed on a surface of the port body 122. As such, rotating the collar 154 about the longitudinal axis can engage the lugs 172 with the helical channel 174 which can urge the collar 154 and cathlock body 152 longitudinally proximally. In an embodiment, the inner surface of the cathlock collar can include a helical channel configured to engage a lug extending radially outward from a portion of the port body 122. As such, rotating the collar can urge the collar 154 and cathlock body 152 longitudinally proximally. In an embodiment, the helical channel 174 can include a recess configured to receive the lug 172 when the cathlock is in the locked position (e.g. FIG. 1D). The recess can receive the lug 172 and prevent any retrograde movement thereof, securing the cathlock 150 in the locked position.

As shown in FIG. 1A, 1C, in an embodiment, the outer surface of port 120 can include a port locking tooth 142 extending from the surface of the port body 122, proximate the port stem 124. The port locking tooth 142 can define a ridge extending parallel to the longitudinal axis and can include one or more sloped or rounded surfaces extending from the ridge to the surface of the port body 122. As shown in FIG. 3C, in an embodiment, an inner surface of the cathlock collar 154 can include a collar locking tooth 144 extending therefrom. The collar locking tooth 144 can define a ridge extending parallel to the longitudinal axis and can include one or more sloped or rounded surfaces extending from the ridge to the surface of the port body 122.

As shown in FIGS. 3F-3G, as the collar 154 is rotated to threadably engage the port, the collar locking tooth 144 can be configured to slide over the port locking tooth 142 in a first rotational direction, and allow the collar to be tightened onto the port body 122. As the collar locking tooth 144 slides over the port locking tooth 142, the interaction can create an audible or tactile indicator, such as a “click.” The audible or tactile indicator can indicate to a clinician that the collar 154 is sufficiently tightened onto the port body 122. In an embodiment, the angle of the sloped surfaces extending from the ridges can be modified to provide increased or reduced resistance between the port locking tooth 142 and the collar locking tooth 144. For example, a shallow angle of a first sloped surface can provide relatively lower resistance, allowing the collar locking tooth 144 to slide over port locking tooth 144 in a first rotational direction with relative ease. A relatively steeper angle of a second sloped surface, opposite the first sloped surface, can provide relatively higher resistance, mitigating the collar locking tooth 144 from sliding over the port locking tooth 144 in a second rotational direction, and preventing retrograde rotation of the collar 154.

As shown in FIG. 3G, the collar locking tooth 144 can engage the port locking tooth 142 in a second rotational direction, opposite to the first rotational direction to mitigate any further movement in the second rotational direction. This can prevent the collar 154 from coming loose after the collar 154 has been tightened on to the port body 122. In an embodiment, a clinician can apply a rotational force to the collar 154 in the second rotational direction to urge the collar locking tooth 144 past the port locking tooth 142 and release the collar 154 from the port body 122.

In an embodiment, the cathlock adapter (“adapter”) 160 is provided as a separate structure and is configured to engage a proximal end of the catheter 110. The adapter 160 can be formed of a substantially rigid, or resilient material. The adapter 160 can include a body 162 and a stem 164, extending from a distal end of the body 162, and configured to engage a lumen 112 of the catheter 110. For example, the adapter 160 can include a first stem 164A configured to engage a first lumen 112A of the catheter 110 and a second stem 164B configured to engage a second lumen 112B of the catheter 110. The outer diameter of at least a portion of the adapter stem 164 can be the same or slightly larger than an interior diameter of the lumen 112 of the catheter 110 such that the stem 164 can engage the lumen 112 of the catheter 110 in an interference fit. In an embodiment, the stem 164 can include an annular protrusion, barb, or similar structure, configured to engage the catheter lumen 112 and retain the catheter 110 with the adapter 160. Advantageously, the annular protrusion can further expand the catheter 110 providing a stronger interference fit therebetween.

A proximal end of the adapter body 162 can be configured to engage a port stem 124. In an embodiment, an outer diameter of the port stem 124 can be the same or slightly smaller than an inner diameter of a lumen 166 of the adapter body 162. As such, the port stem 124 can engage the adapter body 162 with an interference fit, press-fit, or snap fit engagement, or the like. In an embodiment, the port stem 124 can engage the cathlock adapter body 162 in a luer slip fitting engagement. For example, one of the outer profile of the port stem 124, or the inner profile of the cathlock adapter body 162 can define a tapered shape extending at an angle of between 0.5° and 2° from the longitudinal axis. Although greater or lesser angles are also contemplated. As such, the port stem 124 can define a slightly conical or frusto-conical shape. Similarly, the cathlock body 162 can define a tapered lumen shape configured to receive the tapered port stem 124.

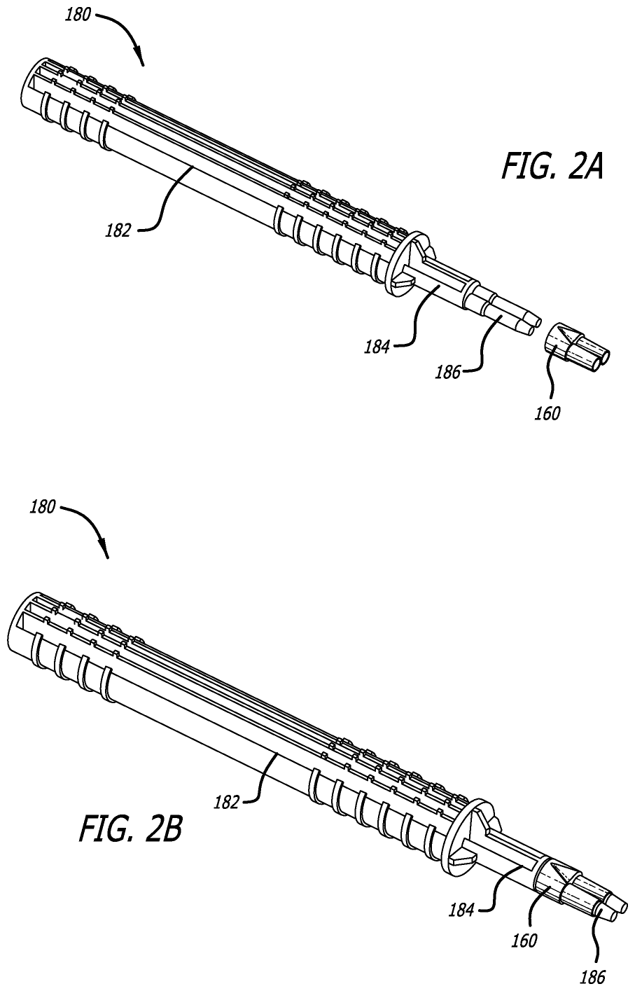

In an embodiment, the system 100 can further include an insertion tool 180 configured to facilitate engaging the adapter 160 with a proximal end of the catheter 110, or facilitate rotating the cathlock collar 154, as described in more detail herein. Advantageously, the insertion tool 180 can facilitate manipulating cathlock 150 or adapter 160 within the confined, wetted environment of the subcutaneous access site, particularly where the components of the system 100 are of a small size, e.g. pediatric sized systems, or for aesthetic reasons. FIGS. 2A-3G show various details of embodiments of the insertion tool 180. As shown in FIGS. 2A-2B, in an embodiment, the insertion tool 180 can include a handle 182 that can define a substantially elongate cylinder. However, it will be appreciated that other shaped handles are also contemplated. The insertion tool 180 can be formed of a substantially rigid material, such as a plastic, polymer, metal, alloy, composite, combinations thereof, of the like. In an embodiment, the handle 182 can include a gripping feature, such as a ridge, rib, abutment, or can include a second material disposed thereon that has an increased friction co-efficient, such as silicone, rubber, polymer, elastomer, or the like.

The handle 182 can include an adapter tool 184 extending therefrom. The adapter tool 184 can be configured to releasably engage the cathlock adapter 160 to facilitate engaging the adapter 160 with a proximal end of the catheter 110. For example, the adapter tool 184 can include one or more adapter protrusions 186 configured to securely but releasably engage a lumen 166 of the cathlock adapter 160. An outer profile of the adapter protrusion 186 can mirror an inner profile of the adapter lumen 166. As such, the adapter protrusion 186 can engage the adapter in a light interference fit, or the like. Further, a distal tip of the protrusion can include a beveled tip to facilitate introducing the adapter tool protrusion 186 into the adapter lumen 166.

In an embodiment, when the adapter 160 is engaged with the adapter tool 184, the beveled tip of the adapter protrusion 186 can extend through the adapter lumen 166 to extend distally of the distal end of the adapter stem 164. As such, when using the insertion tool 180 to urge the adapter 160 onto a proximal end of the catheter 110, the beveled tip can engage the catheter lumen 112 and facilitate stretching an inner diameter of the catheter lumen 112 to an outer diameter of the adapter stem 164. In an embodiment, the adapter tool 184 can include one or more abutment surfaces configured to engage a surface of the adapter 160 and prevent any further proximal movement relative to the insertion tool 180.

In use, the adapter 160 can be slid onto the adapter tool 184, with an adapter protrusion 186 extending through an adapter lumen 186, until an abutment surface engages a surface of the adapter 160. The adapter 160 can be securely retained thereon by a light interference fit, press-fit, or snap fit engagement. A clinician can then manipulate the tool 180 to align the adapter protrusion 186 with a lumen 112 of the catheter 110 and urge the adapter distally onto a proximal end of the catheter 110. The beveled tip of the adapter protrusion 186 can facilitate alignment of the adaptor protrusion 186 with the lumen 112 and/or can stretch the inner diameter of the catheter lumen 112 to an outer diameter of the adapter stem 124 to fit over the adapter protrusion 186 and the adapter stem 164. The clinician can then urge the adapter stem 164 in to the lumen 112 of the catheter 110. In an embodiment, a frictional force between the catheter lumen 112 stretched over the adapter stem 164 can be greater than a frictional force used to retain the adapter 160 on to the adapter tool 184. As such, when the adapter 160 is securely engaged with the catheter (e.g. FIG. 1B), a clinician can withdraw the insertion tool 180 proximally from the catheter 110 which will cause the adapter 160 to disengage the adapter tool 184.

In an embodiment, as shown in FIGS. 3A-3G, the insertion tool 180 can further include a spanner head 190 extending from the handle 182. The spanner head 190 can include a pair of opposing jaws 192, for example a first jaw 192A and a second jaw 192B. Each jaw 192 can define a jaw facet 194 configured to engage a collar facet 158. As such, the jaws 192 of the spanner head 190 can engage the cathlock collar 154. The clinician can then manipulate the handle 192 to provide mechanical advantage to rotate the collar 154, locking the cathlock body 152 with the port body 122.

In an embodiment, as shown in FIGS. 3A-3G, the jaw 192 can include a lip 196 extending across a facet of the jaw 192 and extending inward towards an opposite jaw. The lip 196 can engage a ridge 198 of the collar 154 such that the spanner head 190 can releasably engage the collar 154 securely in a snap-fit engagement. The lip 196 can be configured to engage the collar ridge 198 in one of a first rotational direction, or a second rotational direction opposite the first rotational direction, about the longitudinal axis. In an embodiment, the collar ridge 198 can define an undercut portion configured to securely engage the lip 196 of the jaw 192. Advantageously, the lip 196 can hook on to the ridge 198 to provide improved leverage between the spanner head 190 and the collar 154, and prevent outward expansion and disengagement of the jaws 192 from the collar. Further, the undercut of the ridge does not obstruct the collar facets 158, allowing for other tools, such as hemostats or the like, to grasp the collar 154.

In an embodiment, as shown in FIG. 4D, the port body 122 can further include a gasket 132 disposed annually about the port stem 124. As shown, for example in FIG. 4D, where the port 120 includes a dual lumen port, the port 120 can include a first port stem 124A and a second port stem 124. As such, the gasket 132 can be configured to extend annually about each of the port stems 124A, 124B. The gasket 132 can be disposed between the port body 122 and the catheter adapter 160 to provide a fluid tight seal therebetween when the catheter adapter 160 is engaged with port stem 124.

In an embodiment, as shown in FIG. 4D, the cathlock body 152 can include a ring 134 extending radially inward from an inner wall of the cathlock lumen 156 and extending annularly about the longitudinal axis. The cathlock ring 134 can be configured to abut against a shoulder 168 of the cathlock adapter 160 and urge the cathlock adapter 160 longitudinally proximally on the port stem 124, and optionally the gasket 134. As such, the mechanical advantage employed by the rotation of the collar can urge not only the catheter 110 onto the cathlock adapter stem 164 but can also urge the cathlock adapter 160 onto the port stem 124 and optionally the gasket 134 as well. This can ensure a fluid tight seal between the port 120 and the catheter 110 even when under high pressure infusion.

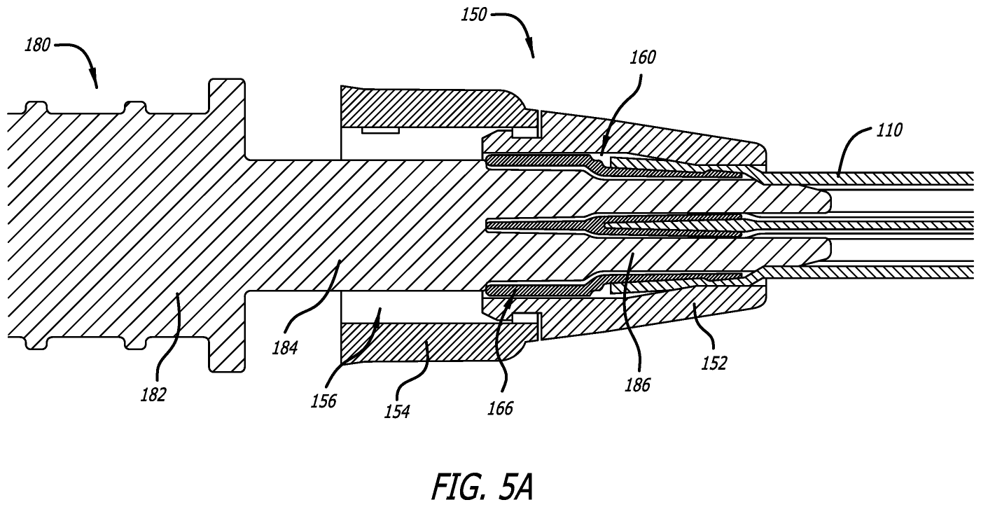

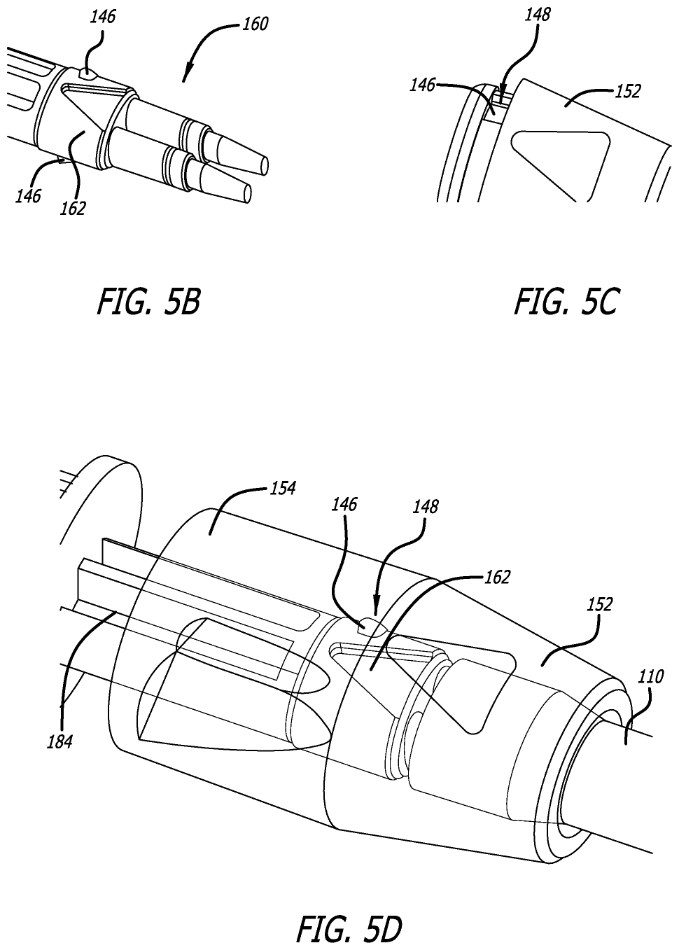

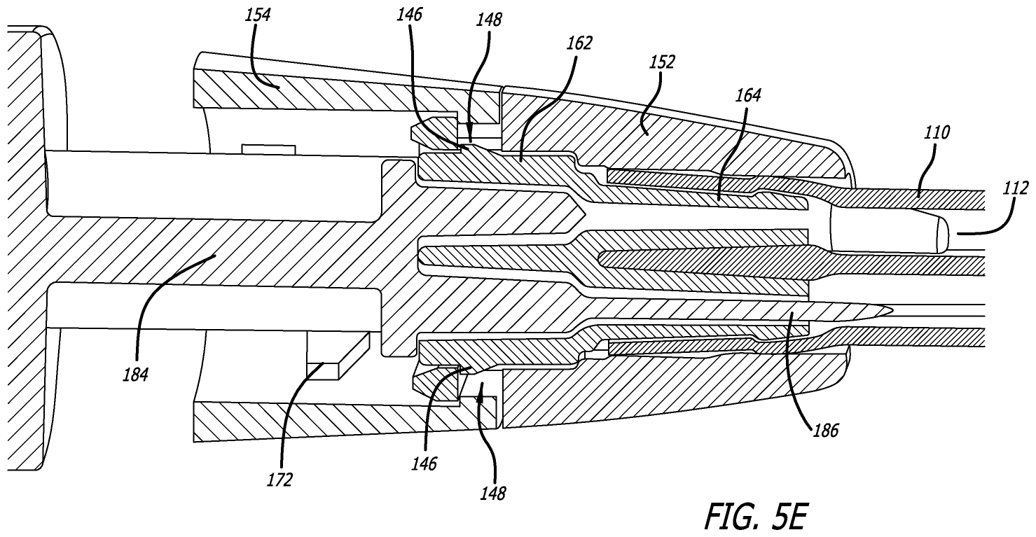

As shown in FIGS. 5A-5E, in an embodiment, the cathlock adapter 160 can be coupled to the cathlock 150 to form a single functional unit. The cathlock adapter 160 can be coupled to the cathlock 150 with one of a press-fit, interference fit, or snap-fit engagement. As shown in FIG. 5A, the adapter tool 184 of the insertion tool 180 can extend through a proximal end of the cathlock lumen 156 so that the adapter protrusions 186 can engage the cathlock adapter lumen 166 of the cathlock adapter body 162. The clinician can then use the insertion tool 180 to manipulate the cathlock adapter 160, as described herein, as well as manipulate the cathlock 150 coupled to the adapter 160.

As shown in FIGS. 5B-5D, in an embodiment, the cathlock adapter 160 engage the cathlock 150 in a snap-fit engagement. FIG. 5B shows the cathlock adapter 160 including a pawl 146 extending from the cathlock adapter body 162. FIG. 5C shows close up detail of the cathlock adapter 160 disposed within the lumen 156 of the cathlock body 152, with the pawl 146 engaged with the recess 148. FIG. 5D shows the cathlock 150 and adapter 160 assembly with the cathlock 150 shown in wire frame. The adapter tool 184 is shown engaged with the lumen 166 of the adapter 160 and the catheter 110 is shown engaged with the adapter stem 164. FIG. 5E shows a cross-section view of the cathlock 150 and adapter 160 assembly, the adapter tool 184 engaged with the lumen 166 of the adapter 160, the catheter 110 engaged with the adapter stem 164, and the pawl 146 engaged with the recess 148.

In an embodiment, the pawl 146 can extend from a side surface of the adaptor body 162. The pawl 146 can include a relatively gently sloping distal surface and a relatively steep proximal surface, each extending from the side surface of the cathlock adapter body 162. In an embodiment, the proximal surface of the pawl 146 can extend substantially perpendicular from the surface of the cathlock adapter body 162.

The pawl 146 can be configured to engage a recess 148 disposed in the cathlock body 152. In an embodiment, the adapter 160 can be engaged with the cathlock 150, for example in an interference fit, and can allow for some longitudinal movement between adapter 160 and the cathlock 150. In an embodiment, the adapter 160 can be a separate structure from the cathlock 150, as described herein, and can be disposed within the cathlock lumen 156 to be slidably engaged the cathlock 150.

In use, the clinician can slide the proximal end of the catheter 110 through the distal end of the cathlock lumen 156 and can urge the cathlock adapter stem 164 in to a proximal end of the catheter lumen 112. When the proximal end of the catheter 110 is fully engaged with the cathlock adapter stem 164, further force applied by the clinician can cause the cathlock adapter 160 to slide relative to the cathlock body 152 such that the pawl 146 can align with the recess 148. The distal surface of the pawl 146 can engage the recess 148 in a snap-fit engagement, and the engagement of the pawl 146 with the recess 148 can provide an audible or tactile indicator, such as a “click”. The pawl 146 can abut against a surface of the cathlock 150 to prevent any proximal movement of the adapter 160 relative to the cathlock 150. Optionally, the snap fit engagement can further lock the catheter 110 to the cathlock adapter stem 164.

Advantageously, the cathlock 150 and adapter 160 assembly can reduce the number of separate parts that a clinician must manipulate. Further, the movement of the cathlock adapter 160 relative to the cathlock body 152, and optionally the snap-fit mechanism, can create an audible “click” and/or tactile signal for the clinician. The audible or tactile signal, can indicate to the clinician that catheter 110 proximal end is fully engaged with the cathlock adapter stem 164. As such the clinician can withdraw the insertion tool 180 from the cathlock lumen 156. Since the insertion tool 180 is engaged with the adapter 160 with a light interference fit, the engagement between the adapter stem 164 and the catheter 110 can cause the adapter 160 to disengage the insertion tool 180 when the insertion tool 180 is withdrawn proximally. The interaction between the pawl 146 engaged with the cathlock can further prevent disengagement of one of the adapter 160 or the cathlock 150 when the insertion tool 180 is withdrawn. With the cathlock adapter 160 and the cathlock 150 assembly engaged with the catheter 110 the clinician can then engage the adapter 160 and cathlock 150 assembly with the port stem 120, as described herein.

In an embodiment, as shown in FIGS. 6A-6C, the cathlock adapter 160 can include a skirt 176 extending distally from the cathlock adapter body 162 and extending annually about the one or more cathlock adapter stem(s) 164. FIG. 6A shows a cross-sectional side view of the catheter adapter 160 including the skirt 176 disposed within a lumen 156 of the cathlock 150. The skirt 176 can define an outer diameter that is substantially the same or slightly less than the inner diameter (d2) of the proximal end of the cathlock lumen 156, but is also greater than the inner diameter (d1) of the distal end of the cathlock lumen 156. The skirt 176 can be formed of the same material as the cathlock adapter 160. In an embodiment, the skirt 176 can be formed of a different material as the cathlock adapter 160, displaying different mechanical properties. The skirt 176 can be resilient enough to maintain a shape while still allowing some deflection, or flexibility if compressed radially inward.

In an embodiment, as shown in FIGS. 6A and 6C, the skirt 176 can form a continuous annular structure extending about the longitudinal axis. In an embodiment, as shown in FIG. 6B, the skirt 176 can include one or more notches extending proximally from a distal end thereof to create a plurality of “fingers” disposed annularly about the one or more cathlock adapter stem(s) 164. The notches can be configured to allow the fingers to flex radially inward and impinge against an outer surface of the catheter 110. The skirt 172 can be configured to compress a portion of the catheter 110 onto the adapter stem 164 and can mitigate tearing or buckling of the catheter 110 as the cathlock 150 is tightened. In an embodiment, the catheter adapter 160, including the skirt 176 can be formed integrally with the port stem 124.

In an embodiment, as shown in FIGS. 6D-6E, an outer surface of the skirt 176, cathlock adapter body 162, or both, can include a threaded structure 178, configured to engage a threaded structure disposed on an inner surface of the cathlock lumen 156. In an embodiment, the outer surface of one of a portion of the port body 122 or the port stem 124 can also include a threaded structure configured to compliment the threaded structure 178 disposed on the outer surface of the skirt 176 or adapter body 162, and also configured to engage the threaded structure disposed on the inner surface of the cathlock lumen 156. Rotating the cathlock collar 154 or cathlock body 152 can threadably engage one of the skirt 176, cathlock adapter body 162, port stem 124 or port body 122. Advantageously, the threaded engagement between the cathlock 150 and the adapter 160 can provide a secure engagement as well as a smooth longitudinal movement therebetween. This engagement can mitigate tearing or buckling of the catheter 110 as the cathlock 150 compresses a portion of the catheter 110 onto the adapter stem 164.

In an exemplary method of use, the proximal end of the catheter 110 can engage the adapter stem 164, with the stem 164 extending into the catheter lumen 112 in an interference fit, as described herein. The catheter 110 can extend over the stem 164 until a proximal tip engages the adapter body 162. The skirt 176 can extend over an outer surface of the catheter 110. The adapter body 162 can then engage the port stem 124 and a cathlock 150 can be urged proximally over the catheter 110 and adapter 160 assembly. A distal portion of the adapter 160 can extend into a proximal portion of the cathlock lumen 156. In an embodiment, the cathlock collar 154 can engage the port 120 and rotate to use mechanical advantage to urge the cathlock body 152 proximally.

In an embodiment, one of the collar 154 or the body 152 of the cathlock 150 can be rotated to threadably engage an outer surface of one of the skirt 176, adapter body 162, port stem 124, or port body 122 and urge the cathlock 150 proximally over the catheter 110 and adapter 160 assembly. At least a portion of the cathlock lumen 156 is tapered from diameter (d2) down to diameter (d1). As such, as the cathlock 150 is urged proximally, the walls of the cathlock lumen 156 compress the skirt 176 radially inward to clamp the catheter 110 between the inner surface of the skirt 176 and the outer surface of the catheter adapter stem 164. Where a cathlock engages a catheter directly and is urged longitudinally proximally, the frictional forces between the cathlock and the catheter can damage or buckle the catheter wall providing a discontinuous annular seal therebetween resulting in fluid leakage. Advantageously, the skirt 176 can provide a radially inward clamping force while mitigating any longitudinal or rotational frictional forces between the cathlock 150 and the catheter 110, and can mitigate any damage to the catheter 110.

In an exemplary method of use, a connection system 100 can be provided, as described herein. In an embodiment, the catheter 110 can be placed within the vasculature of the patient, optionally a proximal end of the catheter 110 can be trimmed to a suitable length. A cathlock 150 body can be slidably engaged with the proximal end of the catheter 110, with the catheter 110 extending through the cathlock lumen 156. In an embodiment, an outer diameter of the catheter 110 can be substantially the same or slightly smaller than an inner diameter (d1) of the distal end of the cathlock lumen 156 so that the cathlock 150 can slidably engage the catheter 110. In an embodiment, the cathlock 150 can frictionally engage the outer surface of the catheter 110 such that the clinician can position the cathlock 150 on the catheter 110 and the cathlock 150 can remain in place without sliding along the catheter 110. Advantageously, the engagement between the cathlock body 152 and the catheter 110 can prevent the cathlock body 152 from sliding freely and sliding too far distally or sliding proximally off of the catheter 110.

The clinician can then couple the adapter 160 with an adapter tool 184 of the insertion tool 180. The clinician can then manipulate the insertion tool 180 to align the adapter 160 with a lumen 112 of the catheter 110 and urge the adapter stem 164 into the catheter lumen 112, as described herein. When the adapter 160 is securely engaged with the catheter lumen 112 the clinician can with withdraw the insertion tool 180 disengaging the adapter tool 184 from the adapter 160, (FIG. 1B). Advantageously, the adapter stem 164 can extend the outer diameter of the proximal end of the catheter 110 to a diameter that is greater than the inner diameter (d1) of the distal end of the cathlock lumen 156. This can prevent the cathlock body 152 from sliding proximally off of the proximal end of the catheter 110.

The clinician can then urge the adapter body 162 onto the port stem 124. As described herein, the port stem 124 can engage the adapter body 162 in an interference fit, press fit, snap fit, or luer slip fit engagement, (FIG. 1C). As shown in FIG. 1D, the cathlock 150 can be slid over the adapter 160 and can threadably engage the port 120. For example, the collar 154 can engage the port body 122 such that the lugs 172 can engage the helical channel 174, as described herein. The clinician can rotate the collar 154 to use mechanical advantage to urge the cathlock 150 longitudinally proximally onto the adapter 160. In an embodiment, the clinician can engage a spanner head 190 of the insertion tool 180 with the collar to provide additional leverage and facilitate rotation of the collar.

As shown the collar 154 includes a facet 158 to engage a facet 194 of the jaws 192 of the spanner head 190. In an embodiment, the collar 154 can include a plurality of the facets to allow the clinician to engage the collar 154 at various angles extending perpendicular to the longitudinal axis. The distal end of the cathlock lumen 156 can engage an outer surface of the catheter 110, proximate the distal end of the adapter stem 164 such that a portion of the catheter 110 can be compressed between the adapter stem 164 and the inner surface of the cathlock lumen 156. As such, the cathlock 150 can ensure a fluid tight seal between catheter 110 and the port 120. Optionally, the cathlock 150 can compress a skirt 176 of the adapter 160 to compress a portion of the catheter 110, as described herein, and ensure a fluid tight seal between catheter 110 and the port 120.

Advantageously, the adapter 160 can be urged longitudinally onto the catheter 110 more easily than urging the catheter 110 directly onto the port stem 124, since the engagement can be performed outside of the access site. The adapter 160 and catheter 110 assembly can then engage the port 120 using the cathlock 150 and the clinician can use mechanical advantage to secure the adapter 160/catheter 110 assembly to the port 120 using the cathlock 150.

In an embodiment, a fluid tight seal is achieved between the catheter 110 and the adapter 160 by compressing the catheter 110 onto the adapter stem 164 using the cathlock 150. The compliant material of the catheter 110 can be stretched slightly to fit over the adapter stem 164. The adapter stem 164 can radially expand the outer diameter of a portion of the catheter 110 to a diameter that is greater than the inner diameter (d1) of the cathlock lumen 156. This can grip the catheter 110 and create a fluid-tight seal as the cathlock 150 is urged longitudinally and compresses the catheter between the cathlock 150 and the stem adapter 160, (e.g. FIG. 4C). Further, the adapter 160, being a separate structure, can be fitted to the catheter 110 outside of the tissue pocket before being coupled to the port 120 that may be already disposed within the tissue pocket and sutured in place. This can allow the clinician to apply more leverage to urge the adapter 160 onto the catheter 110, before coupling the adapter to the port 120, as described herein. Further, the locking teeth 142, 144 can indicate when sufficient torque has been applied to the cathlock to ensure a fluid-tight seal and mitigate and retrograde rotation, causing the cathlock to come loose. Further still, the pawl 146 and recess 144 interaction can indicate when the catheter 110 is fully engaged with the adapter stem 164 also to ensure a fluid-tight seal.

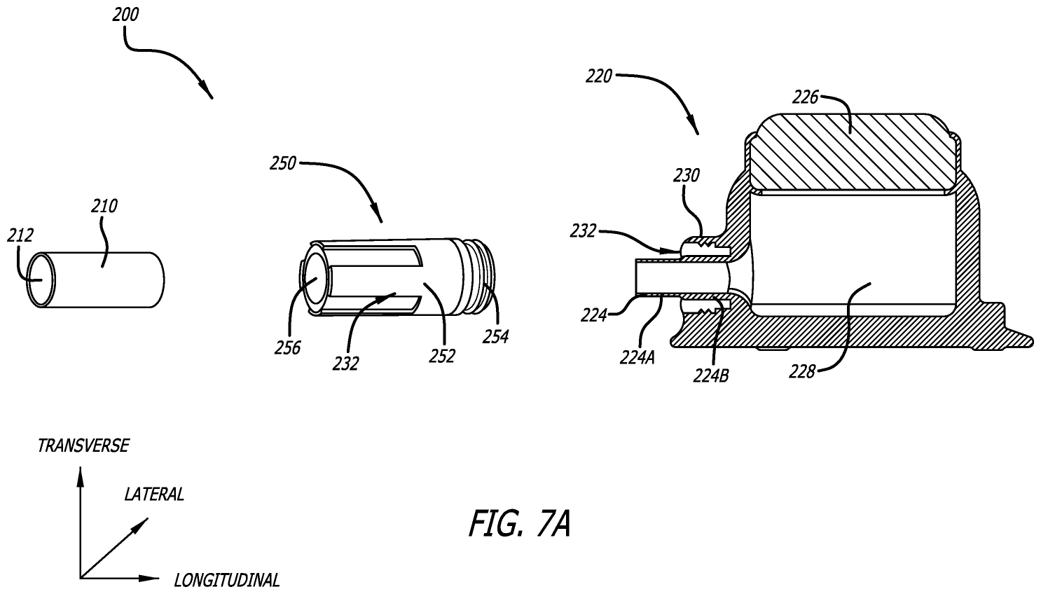

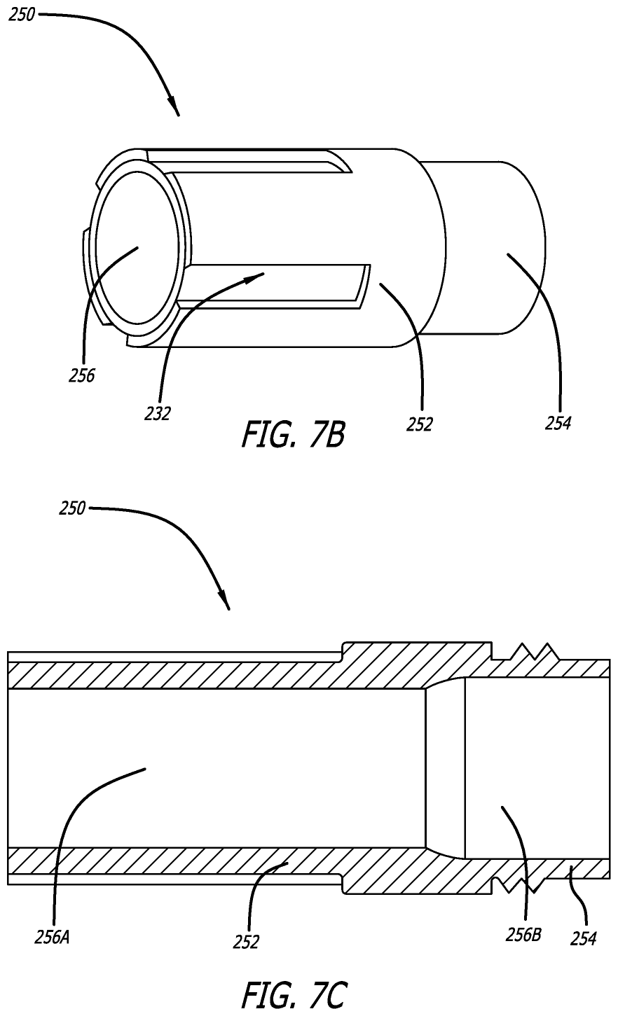

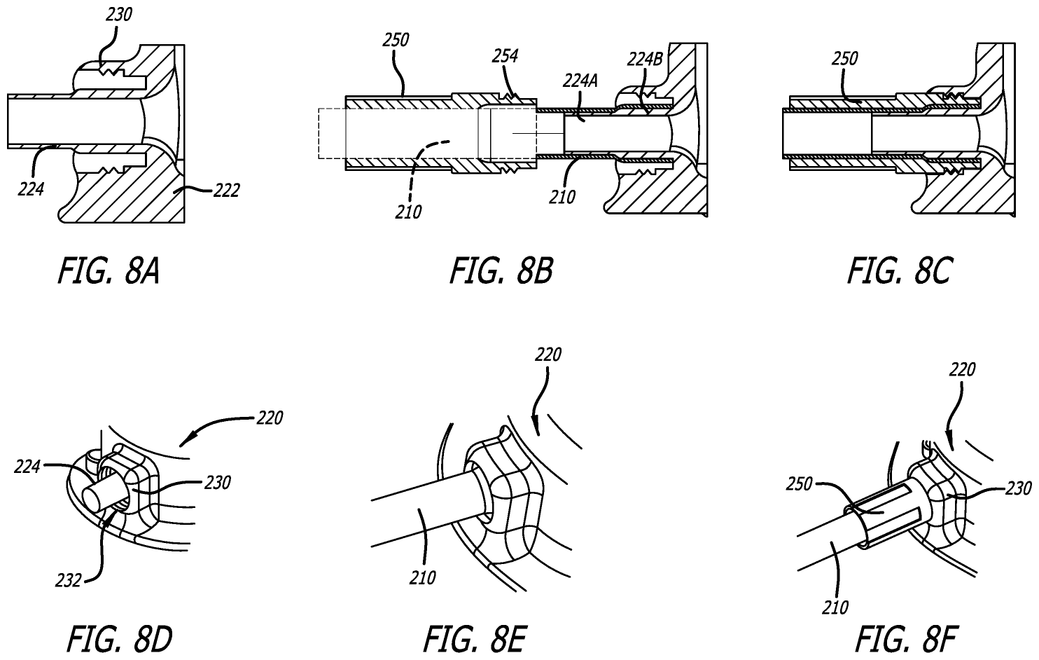

In an embodiment, as shown in FIGS. 7A-8C a connection system 200 can include a catheter 210 defining one or more lumen 212, a port 220 including a port stem 224, and a cathlock 250. In an embodiment, the port can include a port body 222, defining a reservoir 228 that is in fluid communication with the port stem 224. The port 220 can further include a needle penetrable septum 226 disposed over the reservoir 228. It will be appreciated, however, that the port 220 is exemplary, and other configurations of single lumen or multi-lumen, subcutaneous access devices are also contemplated, as described herein.

In an embodiment, the port 220 can include a stem housing 230, surrounding the port stem 224 about an axis thereof. FIGS. 8D-8F show a perspective view of the stem housing 230 surrounding the port stem 224. An outer surface of the stem housing 230 can include one or more facets or gripping features configured to facilitate grasping of the stem housing 230 by a clinician using hemostats, or the like. In an embodiment, the port stem 224 can extend further from the port body 222 than the stem housing 230. In an embodiment, the stem housing 230 can extend further from the port body 222 than the port stem 224. In an embodiment, the port stem 224 and the stem housing 230 can extend equidistant from the port body 222.

As will be appreciated, where the catheter 210 includes two or more lumen 212, the port 220 can include two or more port stems 224 extending from the port body 222 and both encircled by the stem housing 230. In an embodiment, each of the port stems 224 can communicate with separate port reservoirs 228. As used herein, a single lumen catheter 210 and port 220 system is used for ease of explanation but it will be appreciated that multi-lumen catheter and port assemblies are also contemplated to fall within the scope of the present invention.

The stem housing 230 can define an opening 232 that defines an inner diameter sufficient to receive both a portion of the catheter 210 and a portion of the cathlock 250, as described in more detail herein. In an embodiment, an inner surface of the stem housing 230 can include a threaded structure configured to engage a threaded structure disposed on the cathlock 250. In an embodiment, the cathlock 250 can include a body 252 defining a lumen 256 extending along a longitudinal axis and configured to receive the catheter 210 there through. In an embodiment, the inner diameter of the cathlock lumen 265 can be the same or slightly larger than an outer diameter of the catheter 210. In an embodiment, the cathlock lumen 256 can be a smooth bore, or include a low-friction surface or coating, to reduce shearing of the catheter 210 during rotational engagement. In an embodiment, an outer surface of the catheter 210 can include a lubricious coating, or similar low-friction coating to mitigate twisting or shearing of the catheter 210 as the cathlock 250 is tightened, as described in more detail herein.

In an embodiment, the cathlock 250 can include a collar 254 extending longitudinally from the cathlock body 252. The collar 254 can be configured to fit within the opening 232 of the stem housing 230. In an embodiment, the cathlock collar 254 can include a threaded structure configured to threadably engage the threaded structure of the port stem housing 230. In an embodiment, the outer surface of the cathlock body 252 can include a gripping feature configured to facilitate rotation of the cathlock 250 about the longitudinal axis. FIG. 7B shows an alternate embodiment of a cathlock 250 where the cathlock collar 254 does not include a threaded structure and can engage the stem housing 230 in an interference fit, press fit, snap fit, or luer slip fit engagement.

In an embodiment, the catheter lumen 212 can be configured to fit over the port stem 224 in an interference fit, or the like. In an embodiment, the port stem 224 can include a smooth outer surface, or include a low-friction surface or coating, to reduce shearing of the catheter 210 during rotational engagement, as described herein. In an embodiment, the port stem 224 can include a flared portion to provide increased compression of the catheter 210 and/or provide a more secure interference fit between the catheter 210 and the port stem 224. For example, a first portion 224A of the port stem 224 can define an outer diameter that is the same or slightly smaller than an inner diameter of the catheter lumen 212. As such the catheter 210 can be urged over the first portion 224A of the port stem 224 in a relatively light interference fit. A second portion 224B, or “flared portion” of the port stem 224 can define an increase in outer diameter relative to the first portion 224A. The diameter of the second portion 224B can be larger than an inner diameter of the catheter lumen 212 to provide a relatively strong interference fit. In an embodiment, the port stem 224 can include a tapered transition between the first portion 224A and the second portion 224B.

In an embodiment, the cathlock lumen 256 can define a substantially straight walled lumen extending parallel to the longitudinal axis. In an embodiment, as shown in FIG. 7C, a first portion 256A of the cathlock lumen 256 can define a first diameter and a second portion 256B of the cathlock lumen 256 can define a second diameter larger than the first diameter. The second portion 256B of the cathlock lumen 256 and can align with the flared portion 224B of the port stem 224B. In an embodiment, the cathlock lumen 256 can include a tapered portion providing a transition between the first portion 256A and the second portion 256B.

FIGS. 8A-8C show an exemplary method of use for coupling the catheter 210 to the port 220 using the cathlock 250 and the stem housing 230. A port 220 is provided including a port stem 224 and a stem housing 230 as described herein. Optionally a proximal end of a catheter 210 can be trimmed to a suitable length. The catheter 210 can then be threaded through the lumen 256 of the cathlock 250 and a proximal end of the catheter 210 can engage the port stem 224. As described herein the catheter 210 can engage the port stem 224 in an interference fit, or the like.

The cathlock 250 can then slide over the catheter 210 towards the port 220 until the collar 254 engages the opening 232 of the stem housing 230. Urging the cathlock 250 into the stem housing opening 232 can compress the proximal portion of catheter 210 between the cathlock 250 and the port stem 224. The cathlock 250 can engage the stem housing 230 in a luer slip fitting engagement to secure the cathlock 250 and the catheter to the port 220. In an embodiment, the cathlock 250 can threadably engage the stem housing 230 and use mechanical advantage to urge the cathlock collar 254 into stem housing 230 and secure the cathlock 250 thereto.

In an embodiment, the flared portion of the port stem, i.e. second portion 224B can provide an increased pressure on the catheter 210, between the port stem 224 and the cathlock 250, as the cathlock collar 254 advances over the catheter 210 disposed on the flared portion of the port stem 224. This can provide an increased pressure and an improved seal between the catheter 210 and the port 220. Advantageously, the cathlock 250 provides a secure connection between the catheter 210 and the port stem 224 while mitigating leakage from the connection, even under high infusion pressure. Further the cathlock 250 mitigates tearing or shearing of the catheter 210 during the connection process.

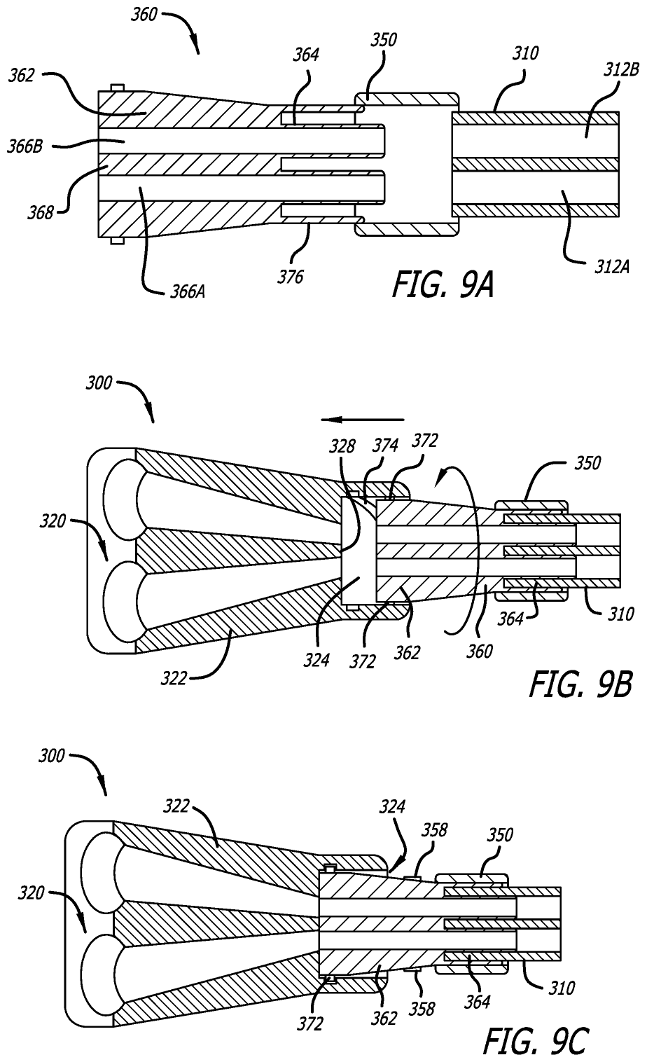

FIGS. 9A-9C show an embodiment of a connection system 300, including a catheter 310 defining one or more lumen 312, a cathlock 350, an adapter 360, and a port 320 including a port body 322 defining a port socket 324. The adapter 360 includes an adapter body 362 defining a substantially circular cross-sectional shape, and an adapter stem 364, extending longitudinally therefrom. The adapter body 362 and adapter stem 364 can define a lumen 366 that provides fluid communication between the catheter 310 and the port 320.

The adapter stem 364 can define an outer diameter that is substantially the same, or slightly larger than an inner diameter of the catheter lumen 312. As such, the adapter stem 364 can be urged into the catheter lumen 312 to engage therewith in an interference fit. In an embodiment, the adapter 360 can further include a skirt 376 extending longitudinally from the adapter body 362 and encircling a portion of the adapter stem 364 about the longitudinal axis. The skirt 376 can be formed of a resilient material and can be flexibly deformed radially inward to compress a portion of the catheter 310 between the skirt 376 and the outer surface of the adapter stem 364. In an embodiment, a cathlock 350 can slidably engage an outer surface of the catheter 310 and can compress the catheter 310 onto the adapter stem 364 to secure the catheter 310 thereto. In an embodiment, the cathlock 350 can compress the skirt 376 onto the catheter 310 and the adapter stem 364 securing the catheter 310 thereto.

In an embodiment, the port 320 can include a socket 324 that defines a recess configured to receive a portion of the adapter body 362 therein. The adapter body 362 can rotatably engage the socket 324 to lock the adapter 360 thereto and provide fluid communication between the port 320 and the adapter lumen 366. In an embodiment, the adapter body 362 can threadably engage the port socket 324. In an embodiment, the catheter body 362 can include one or more lugs 372 configured to engage a helical channel 374 disposed on an inner surface of the port socket 324. As the catheter body 362 is rotated, the lugs 372 can engage the helical channel 374 to urge the adapter body 362 longitudinally into the port socket 324 until an adapter engagement surface 368 of the adapter body 362 engages a port engagement surface 328 to provide fluid communication between the port 320 and the adapter 360 (FIG. 9C). In an embodiment, an outer surface of the adapter 360 can include a facet 358 or similar gripping feature configured to facilitate rotation of the adapter body 362 about the longitudinal axis. In an embodiment, the facet 358 can be configured to engage a spanner head 190 of an insertion tool 180 as described herein.

In an exemplary method of use, a clinician can position a catheter 310 within the vasculature of the patient, with a distal tip of the catheter 310 at a target location. Optionally, a proximal end of the catheter 310 can be trimmed to a suitable length. A clinician can then urge an adapter 360 onto the proximal end of the catheter 310 by urging the adapter stem 364 into a lumen 312 of the catheter 310 and engaging therewith in an interference fit.

In an embodiment, the clinician can then slide a cathlock 350 over the portion of the catheter 310 that engages these adapter stem 364 to compress the catheter 310 onto the adapter stem 364 securing the catheter 310 thereto. In an embodiment, the adapter 360 can include a skirt 376. The proximal end of the catheter 310 can extend between the adapter stem 364 and the skirt 376. The cathlock 350 can then be slid over the skirt 376, compressing the skirt 376 radially inward to clamp the catheter 310 onto the adapter stem 364. Advantageously, the skirt 376 can prevent sheering or tearing of the catheter 310 as the cathlock 350 is tightened about the catheter 310, as described herein.

The clinician can then engage the adapter body 362 with the port socket 324 and rotate the adapter body 362 about the longitudinal axis to urge the adapter 360 longitudinally onto the port 320, securing thereto. In an embodiment, a lug 372 can engage a helical channel 374. As the adapter body 362 is rotated, the mechanical advantage of the lug 372 engaging the helical channel 374 can urge the adapter 360 onto the port 320. In an embodiment, the port 320 can include a gasket disposed between the adapter engagement surface 368 and the port engagement surface 328 to ensure a fluid-tight seal therebetween. In an embodiment, a clinician can use an insertion tool 180 to facilitate coupling the adapter 360 with the catheter 310 or rotating the adapter 360 to engage the port socket 324, as described herein.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A connection system for a subcutaneous port including a port stem, the connection system comprising:
an adapter having a body and configured for connection to the port stem, and an adapter stem extending from the body and configured for insertion into a lumen of a catheter to connect the adapter to the catheter; and
a cathlock including a lumen and configured for sliding over an outer surface of the catheter, the cathlock having threading for engagement with the port stem, wherein engaging the cathlock to the port stem compresses the catheter radially inward onto the adapter.

2. The connection system according to claim 1, wherein the adapter stem is configured to radially expand the catheter lumen to secure the adapter to the catheter in an interference fit.

3. The connection system according to claim 1, wherein the adapter is formed of a resilient material.

4. The connection system according to claim 1, wherein the adapter body engages the port stem in one of an interference fit, press fit, snap-fit, or luer-slip fit engagement.

5. The connection system according to claim 1, wherein the adapter includes a skirt extending longitudinally from the body and disposed annularly about the adapter stem, the skirt configured to elastically deform radially inward to engage the outer surface of the catheter.

6. The connection system according to claim 5, wherein the skirt is formed of a plurality of fingers extending longitudinally from the body of the adapter and disposed annularly about the adapter stem, the plurality of fingers configured to elastically deform radially inward to engage the outer surface of the catheter.

7. The connection system according to claim 6, wherein a tip of a finger of the plurality of fingers includes a protrusion extending radially inward and configured to engage a portion of the catheter.

8. The connection system according to claim 5, wherein an outer surface of the skirt includes a threaded portion configured to threadably engage the cathlock.

9. The connection system according to claim 1, wherein the cathlock includes a body rotatably coupled to a collar, the collar including a lug configured to engage a helical channel disposed on the port, and wherein rotating the collar urges the lug through the helical channel and urges the cathlock in a longitudinal direction.

10. The connection system according to claim 9, wherein an inner surface of the collar includes a collar locking tooth configured to engage a port locking tooth disposed on the port and to provide an audible or tactile indicator, or to prevent retrograde rotation of the collar.

11. The connection system according to claim 10, wherein the cathlock body and collar define the cathlock lumen, a lumen diameter at the body includes a first diameter and a lumen diameter at the collar defines a second diameter, larger than the first diameter, the lumen including a tapered portion extending from the second diameter to the first diameter.

12. The connection system according to claim 11, wherein a portion of the catheter disposed on the adapter stem defines an outer diameter that is greater than the first diameter of the cathlock lumen and less than the second diameter of the cathlock lumen.

13. The connection system according to claim 1, wherein the cathlock lumen includes a ring extending radially inward from an inner wall thereof, the ring configured to abut against a shoulder of the adapter.

14. The connection system according to claim 1, wherein the port includes a gasket disposed between the port and the adapter and encircling the port stem.

15. The connection system according to claim 1, further including an insertion tool having a handle, an adapter tool configured to engage a lumen of the adapter, or a spanner head configured to engage the collar of the cathlock.

16. The connection system according to claim 15, wherein the spanner head of the insertion tool includes a jaw configured to engage a facet of the collar and to facilitate rotation of the collar about the longitudinal axis.

17. The connection system according to claim 16, wherein the jaw includes a lip configured to engage an undercut of a collar ridge of the collar and to facilitate rotation of the collar about the longitudinal axis.

18. The connection system according to claim 1, wherein a proximal end of the catheter is trimmable.

19. The connection system according to claim 1, wherein the adapter includes a pawl configured to engage a recess disposed in the cathlock in a snap-fit engagement to provide an audible or tactile signal that the lumen of the catheter is fully engaged with the adapter.

20. The connection system according to claim 1, wherein the port includes a port locking tooth, and the cathlock includes a cathlock locking tooth, extending from a surface of the cathlock lumen, the port locking tooth configured to engage the cathlock locking tooth in a snap-fit engagement to mitigate retrograde rotation of the cathlock.

21-50. (canceled)
